(19) United States
(12) Patent Application Publication
(10) Pub. No.: US 2022/0192876 A1
(43) Pub. Date: Jun. 23, 2022

(54) MODULE FOR ASPIRATION AND IRRIGATION CONTROL

(71) Applicant: JOHNSON & JOHNSON SURGICAL VISION, INC., Santa Ana, CA (US)

(72) Inventors: Yehuda Algawi, Binyamina (IL); Assaf Govari, Haifa (IL); Eran Aharon, Haifa (IL); Gili Attias, Haifa (IL); Ilya Sitnitsky, Nahariya (IL); Stanislav Katzir, Hadera (IL)

(21) Appl. No.: 17/130,409

(22) Filed: Dec. 22, 2020

(51) Int. Cl. A61F 9/007; A61M 3/02

(52) U.S. Cl. CPC A61F 9/00745; A61M 3/0283; A61M 2210/0612; A61M 2205/3334; A61M 2205/3327; A61M 2205/3331; A61M 2205/52

(57) ABSTRACT

A module for controlling irrigation and aspiration of a phacoemulsification probe inserted into an eye includes an irrigation link, an aspiration link, a bypass channel, an aspiration valve, a diversion valve, a first and second sensors and a processor. The first sensor and second sensor are configured to measure fluid parameters in the irrigation link and in the aspiration link. The processor is in communication with the sensors, and is configured to identify a change in at least one of the fluid parameters by reading at least one of the first sensor and the second sensor, and, in response to the identified change in the at least one of the fluid parameters, (i) close the aspiration valve and (ii) maintain a pressure of the irrigation fluid delivered to the probe within a predefined range, by regulating the fluid flow via the bypass channel using the diversion valve.

### FIELD OF THE INVENTION

The present invention relates generally to phacoemulsification systems and probes, and particularly to modules for aspiration and irrigation control.

### BACKGROUND OF THE INVENTION

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution (BSS) to maintain the anterior chamber of the eye. After removing the cataract with phacoemulsification, the softer outer lens cortex is removed with suction. An intraocular lens (IOL) is then introduced into the empty lens capsule restoring the patient's vision.

Various techniques of irrigation and aspiration control with medical probes were proposed in the patent literature. For example, U.S. Patent Application Publication 2019/0282401 describes various arrangements of fluidics systems. In one arrangement, an aspiration circuit for a fluidics system is disclosed that selectively controls aspiration. The aspiration circuit comprises an aspiration line operatively connected to a surgical instrument, an aspiration exhaust line operatively connected to a waste receptacle; an aspiration vent line connected at a first end to the aspiration line; and a selectively variable vent valve operatively connected to the aspiration vent line. The variable vent valve may be selectively moved to vary aspiration pressure within the aspiration line. Other fluidics systems are disclosed that include a selectively positionable irrigation valve that may also be incorporated into a fluidics system that includes a variable vent valve.

As another example, U.S. Patent Application Publication 2019/0262175 describes a system, including a handpiece with a tool formed by a hollow needle forming a first channel, a second channel formed between the hollow needle and an enveloping lateral surface, an irrigation device, an aspiration device, a manifold device and a control device. In a first operating mode, the manifold device connects the second channel and the irrigation device for the exchange of fluids and connects the first channel and the aspiration device for the exchange of fluids. In a second operating mode, the manifold device connects the first channel and the irrigation device for the exchange of fluids. The pressure at which the irrigation device delivers fluid to the manifold device is controllable linearly in the second operating mode by the control device.

U.S. Patent Application Publication 2015/0359666 describes a cyclic aperture flow regulator system to control flow exiting from a body cavity during surgery in a way that post-occlusion surges are effectively suppressed. The system is composed by an adjustable fluid aperture installed in a fluid path connecting the aspiration port of a surgical probe with a vacuum source, the probe to be inserted in a body cavity. The cross-sectional area of the fluid aperture can be modified by a cyclic action of an actuator portion driven by a controller, such that a fluid aperture cross-sectional area is modulated. This optionally include a transient complete closure of the aperture. Flow rate across the cyclic aperture flow regulator system can be regulated.

### SUMMARY OF THE INVENTION

An embodiment of the present invention that is described hereinafter provides a module for controlling irrigation and aspiration of a phacoemulsification probe inserted into an eye, the module including an irrigation link, an aspiration link, a bypass channel, an aspiration valve, a diversion valve, a first and second sensors and a processor. The irrigation and aspiration links are configured to be coupled, respectively, with irrigation and aspiration lines and with an irrigation and aspiration channels of the probe. The bypass channel is coupled with the irrigation link and the aspiration link to enable diversion of irrigation fluid from the irrigation link to the aspiration link. The aspiration valve is coupled with the aspiration link to regulate fluid flow via the aspiration link. The diversion valve is coupled with the bypass channel to regulate fluid flow from the irrigation link to the aspiration link. The first and second sensors are coupled, respectively, with the irrigation and the aspiration links, wherein the first sensor and second sensor are configured to measure fluid parameters in the irrigation link and in the aspiration link. The processor is in communication with the first sensor and the second sensor, and is configured to identify a change in at least one of the fluid parameters by reading at least one of the first sensor and the second sensor, and, in response to the identified change in the at least one of the fluid parameters, (i) close the aspiration valve and (ii) maintain a pressure of the irrigation fluid delivered to the irrigation channel within a predefined range, by regulating the fluid flow via the bypass channel using the diversion valve.

In some embodiments, at least one of the fluid parameters are selected from the group consisting of vacuum, pressure, and flow, and the change of the at least one of the fluid parameters indicates an aspiration blockage or a release of the aspiration blockage.

In some embodiments, the module further includes connectors for detachably connecting the irrigation line and the aspiration line to the phacoemulsification probe.

In an embodiment, the module further includes a package containing the irrigation link, the aspiration link, the bypass channel, the aspiration valve, the diversion valve, the sensors and the processor.

In some embodiments, the processor is configured to identify the change in the at least one of the fluid parameters being an increase in vacuum or pressure in the aspiration line or aspiration channel.

In some embodiments, the processor is configured to adjust the diversion valve so as to maintain a pressure level in the irrigation channel within a predefined limit.

In an embodiment, the first sensor includes a pressure sensor coupled with the irrigation link distally to the bypass channel, the second sensor includes a vacuum sensor coupled with the aspiration link distally to the bypass channel, and wherein the module further includes a third sensor, wherein the third sensor is a flow sensor coupled with the aspiration link proximally to the bypass channel.

In another embodiment, the diversion valve is a variably rotatable valve including (i) a lever coupled with the diversion valve, and (ii) one or more solenoid pistons that, when actuated by the processor, moves the lever so as to generate rotational torque causing the diversion valve to adjust an opening in the bypass channel.

There is additionally provided, in accordance with another embodiment of the present invention, a module for controlling irrigation and aspiration of a phacoemulsification probe inserted into an eye, the module including an irrigation link, an aspiration link, a bypass channel, a diversion valve, a first and second sensors and a processor. The irrigation and aspiration links are configured to be coupled, respectively, with irrigation and aspiration lines and with an irrigation and aspiration channels of the probe. The bypass channel is coupled with the irrigation link and the aspiration link to enable diversion of irrigation fluid from the irrigation link to the aspiration link. The diversion valve is coupled with the bypass channel to regulate fluid flow from the irrigation link to the aspiration link. The first and second sensors are coupled, respectively, with the irrigation and the aspiration links, wherein the first sensor and second sensor are configured to measure fluid parameters in the irrigation link and in the aspiration link. The processor is in communication with the first sensor and the second sensor, and in communication with an ultrasonic power source of the phacoemulsification probe, wherein the processor is configured to identify a change in at least one of the fluid parameters by reading at least one of the first sensor and the second sensor, and, in response to the identified change in the at least one of the fluid parameters, (i) activate the diversion valve to regulate the fluid flow via the bypass channel and (ii) adjust the ultrasonic power source of the phacoemulsification probe.

In some embodiments, the at least one of the fluid parameters are selected from the group consisting of vacuum, pressure, and flow, and the change of the at least one of the fluid parameters indicates an aspiration blockage or a release of the aspiration blockage.

In some embodiments, the processor is configured to adjust the ultrasonic power by shutting off the power. In other embodiments, the processor is configured to adjust the ultrasonic power by changing one or more selected from the group consisting of frequency, duty cycle, and vibration mode of the probe.

In an embodiment, the module further includes connectors for detachably connecting the irrigation line and the aspiration line to the phacoemulsification probe.

In another embodiment, the module further includes a package containing the irrigation link, the aspiration link, the bypass channel, an electrical link for communication with the ultrasonic power source, the diversion valve, the sensors and the processor.

In some embodiments, the processor is configured to identify the change in aspiration, and to control the diversion valve.

In some embodiments, the processor is configured to adjust the diversion valve so as to maintain a pressure level in the irrigation channel within a predefined limit.

In an embodiment, the first sensor includes a pressure sensor coupled with the irrigation link distally to the bypass channel, the second sensor includes a vacuum sensor coupled with the aspiration link distally to the bypass channel, and wherein the module further includes a third sensor, wherein the third sensor is a flow sensor coupled with the aspiration link proximally to the bypass channel.

In some embodiments, the diversion valve is a variably rotatable valve including (i) a lever coupled with the diversion valve, and (ii) one or more solenoid pistons that, when actuated by the processor, moves the lever so as to generate rotational torque causing the diversion valve to adjust an opening in the bypass channel.

There is further provided, in accordance with another embodiment of the present invention, a method of controlling irrigation and aspiration of a phacoemulsification probe inserted into an eye, the method including providing a module configured to control the irrigation and aspiration of the phacoemulsification probe, wherein the module includes (a) an irrigation link, configured to be coupled with an irrigation line and an irrigation channel of the phacoemulsification probe, (b) an aspiration link, configured to be coupled with an aspiration line and an aspiration channel of the phacoemulsification probe, (c) a bypass channel coupled with the irrigation link and the aspiration link to enable diversion of irrigation fluid from the irrigation link to the aspiration link, (d) an aspiration valve coupled with the aspiration link and configured to regulate fluid flow via the aspiration link, (e) a diversion valve coupled with the bypass channel and configured to regulate fluid flow from the irrigation link to the aspiration link, (f) a first sensor coupled with the irrigation link and a second sensor coupled with the aspiration link, wherein the first sensor and second sensor are configured to measure fluid parameters in the irrigation link and in the aspiration link, and (g) a processor in communication with the first sensor and the second sensor, wherein the processor is configured to identify a change in at least one of the fluid parameters. At least one of the first sensor and the second sensor are read to identify a change in the at least one of the fluid parameters. In response to the identified change in the at least one of the fluid parameters, (i) the aspiration valve is closed, and (ii) a pressure of the irrigation fluid delivered to the irrigation channel is maintained within a predefined range, by regulating the fluid flow via the bypass channel using the diversion valve.

In some embodiments, the at least one of the fluid parameters are selected from the group consisting of vacuum, pressure, and flow, and the identified change of the at least one of the fluid parameters indicates an aspiration blockage or a release of the aspiration blockage.

In some embodiments, the identified change in the at least one of the fluid parameters is an increase in vacuum or pressure in the aspiration line or aspiration channel.

There is further yet provided, in accordance with another embodiment of the present invention, a method of controlling irrigation and aspiration of a phacoemulsification probe inserted into an eye, the method including providing a module configured to control the irrigation and aspiration of the phacoemulsification probe, wherein the module includes (a) an irrigation link, configured to be coupled with an irrigation line and an irrigation channel of the phacoemulsification probe, (b) an aspiration link, configured to be coupled with an aspiration line and an aspiration channel of the phacoemulsification probe, (c) a bypass channel coupled with the irrigation link and the aspiration link to enable diversion of irrigation fluid from the irrigation link to the aspiration link, (d) a diversion valve coupled with the bypass channel and configured to regulate fluid flow from the irrigation link to the aspiration link, (e) a first sensor coupled with the irrigation link and a second sensor coupled with the aspiration link, wherein the first sensor and second sensor are configured to each measure a fluid parameter in the irrigation link and in the aspiration link, and (f) a processor in communication with the first sensor and the second sensor, and in communication with an ultrasonic power source of the phacoemulsification probe, wherein the processor is configured to identify a change in at least one of the fluid parameters. At least one of the first sensor and the second sensor are read to identify a change in the at least one of the fluid parameters. In response to the identified change in the at least one of the fluid parameters, (i) the diversion valve is activated to regulate the fluid flow via the bypass channel, and (ii) the ultrasonic power source of the phacoemulsification probe is adjusted.

The present invention will be more fully understood from the following detailed description of the embodiments thereof, taken together with the drawings in which:

### BRIEF DESCRIPTION OF THE DRAWINGS

FIG. 1 is a schematic, pictorial view, along with an orthographic side view, of a phacoemulsification apparatus comprising an aspiration and irrigation control module for aspiration and irrigation control, in accordance with an embodiment of the present invention;

FIG. 2 is a schematic block diagram of the aspiration and irrigation control module of FIG. 1, in accordance with an embodiment of the present invention;

FIG. 3 is a schematic, pictorial cross-sectional view of the variable valve of the aspiration and irrigation control module of FIG. 2, in accordance with an embodiment of the present invention;

FIG. 4 is a flow chart schematically illustrating a method for overcoming a vacuum surge using the aspiration and irrigation control module of FIG. 2, in accordance with an embodiment of the present invention;

FIG. 5 is a flow chart schematically illustrating a method of manufacturing the aspiration and irrigation control module of FIG. 2, in accordance with an embodiment of the present invention;

FIG. 6 is a schematic block diagram of an aspiration and irrigation control module, in accordance with another embodiment of the present invention; and

FIG. 7 is a flow chart schematically illustrating a method for overcoming a vacuum surge using the aspiration and irrigation control module of FIG. 6, in accordance with another embodiment of the present invention.

### DETAILED DESCRIPTION OF EMBODIMENTS

### Overview

During phacoemulsification of a cataracted eye lens, the emulsified lens particles are aspirated. When a particle blocks the inlet of the aspiration channel the vacuum in the line increases. When the line later becomes unblocked (e.g., when the particle is subsequently sucked into the line), the high vacuum in the line causes an aspiration surge with potentially traumatic consequences to the eye.

A possible solution to the problem of vacuum level surge is an aspiration bypass. Such a bypass may consist of a small hole or channel between the irrigation channel and the aspiration channel. When blockage occurs, the high vacuum diverts irrigation fluid into the aspiration channel via the hole, thereby limiting the vacuum level.

In the context of this description a blockage may be either a complete one, or a partial blockage.

However, the above-described bypass aspiration technique is still prone to produce a traumatic aspiration surge when the aspiration line unblocks, since the high vacuum is present in a long line between a portion of the aspiration channel inside the emulsification probe and the aspiration pump. This large volume, partially vacant or filled with liquid, may therefore cause a surge. Moreover, diversion of irrigation fluid from the irrigation line to the aspiration line may “starve” the eye of irrigation fluid, or even create a vacuum in the irrigation line. Either of these actions is potentially traumatic.

Embodiments of the present invention that are described hereinafter provide standalone disposable detachable add-on modules for aspiration and irrigation control to reduce risks from irregular performance of aspiration and/or irrigation.

In some embodiments, a disposable module (e.g., for one-time use) is configured to be inserted between a phacoemulsification probe and the irrigation and aspiration lines. The module comprises an aspiration link that is inserted between the aspiration line and the aspiration channel of the probe, an irrigation link that is inserted between the irrigation line and the irrigation channel of the probe, and a bypass channel that connects between the irrigation link and the aspiration link within the module. The module further comprises a set of valves, including (i) a diversion valve on the bypass channel and (ii) an aspiration valve on the aspiration link distally to the bypass channel. The module additionally comprises a set of sensors, e.g., flow, pressure or vacuum sensors, and a processor that controls the set of valves of the module based on the sensor readings. By controlling the valves, the processor is configured to control the flows in the aspiration link, the irrigation link and the bypass channel, in order to maintain flow, pressure and/or vacuum readings from the sensors within predefined limits.

In some embodiments, the diversion valve and the aspiration valve are fast-acting valves, both controlled by the processor. The valves and the processor (which may be battery operated) may all be contained in a detachable, disposable package (e.g., a case). The diversion valve may be a variable flow valve, which is placed in the irrigation diversion (bypass) channel. The aspiration valves may be an on/off valve, which is placed in the aspiration line, distally to the bypass channel. In an embodiment, the aspiration valve may be a variable flow valve. The processor receives signals from sensors coupled to the irrigation and aspiration lines passing inside the package. In response to the signals, the processor activates each of the valves to adjust vacuum, pressures and/or flow rates.

During normal operation, the diversion valve is closed and the aspiration valve is open. If the processor detects a blockage in the aspiration line or detects the occlusion is being released (i.e., aspiration blockage or release of aspiration blockage), it closes the aspiration valve and opens the diversion valve, but continues to monitor the irrigation line. If there is too much reduction in irrigation flow, the processor reduces the diversion or flow provided by the diversion valve.

The sequence of opening and closing the two valves, as well as varying of the rate of diversion, is governed by an algorithm operated by the processor. The algorithm operates according to preset acceptable limits for the values read by the sensors, i.e., for the flow rate of the irrigation fluid and the vacuum in the aspiration line.

Typically, the processor operates the two valves in coordination to allow aspiration capacity that varies between a full flow of aspirated fluid with no irrigation fluid diversion into the aspiration line and, in case of a blockage of and/or an occlusion released in the aspiration line, complete diversion of the irrigation fluid. Between these states, the valve(s) is controlled (e.g., rotated) by the processor in an intermediate regime that maintains the pressures within desired ranges.

Moving the bypass channel from a fixed opening in the tip to a valve-controlled channel proximal to the handle has considerable benefits for reducing vacuum surge. Using valves to simultaneously disconnect or alter the fluid flow to/from the eye and the aspiration line and divert the irrigation into that line means that the only vacuum that can affect the eye is in the short section of the aspiration line within the probe (between the valve and the eye). In contrast, when using an opening in the tip for bypass, the vacuum in the entire length of the aspiration line, all the way to the console, typically five to six feet long, affects the eye. As response time to a vacuum surge is largely proportional to the vacant volume, placing a valve in the handle reduces the response time considerably (e.g., from tens of mSec to several mSec), and therefore reduces the risk of eye trauma.

As the disclosed module containing the variable valve is designed to be disposable, the valve components should be as inexpensive as possible. However, some low-cost valves, such as certain rotatable valves, may sometimes seize or jam, and do not rotate completely, because of the material in the aspiration line. Thus, in one embodiment, a lever is attached to the variable valve, the two ends of the lever having embedded magnets with solenoids on either side of each magnet. With this arrangement, by appropriate selection of the currents activating the solenoids, a very strong leveraged force, capable of overcoming any valve seizure, can be applied. By switching the solenoid currents the force can be applied to either open or close the valve. The disclosed disposable module is standalone in the sense that they do not require any sort of information or control signals from any external entity in order to control the valves. To this end, in some embodiments, all data required by the processor of the module is provided by the sensors that are internal to the module, as well. In other words, the processor is configured to identify the aspiration blockage or the release of aspiration blockage, and to control the aspiration valve and the diversion valve, based only on readings of the sensors in the module.

Another embodiment of the disposable module is provided, to answer a scenario in which particles in the aspiration line prevent an aspiration valve from closing completely. In this embodiment, the module only has a single diversion valve. In this embodiment, the module processor has an electrical link to the system, to enable the processor.

Using the electrical link, the processor may, in addition to adjusting the diversion valve, command other elements of the system to assist in fluid regulation, as described below.

In this another embodiment, while there is no occlusion in the aspiration line, the diversion valve is closed. When the processor (e.g., of the module) detects an occlusion in the aspiration line from the sensors in the line, the processor opens the diversion valve, so diverting some of the irrigation fluid into the aspiration line, and thus preventing the vacuum in the aspiration line from becoming stronger.

At the same time, the processor of the console typically, using the electrical line to the console, commands turning off the ultrasound (US) to the emulsifying needle, to prevent overheating of the eye, and may also increase the irrigation rate to compensate for the diverted irrigation fluid.

When the processor detects the occlusion has cleared, the processor closes the diversion valve and turns the emulsifying needle back on. By not having any valve in the aspiration line, there is no possibility of such a valve functioning incorrectly.

By providing a standalone disposable module capable of dynamic bypass aspiration control, the disclosed embodiments of the invention may improve the safety and efficacy of phacoemulsification procedures, using, for example, existing probes and phacoemulsification systems.

### System Description

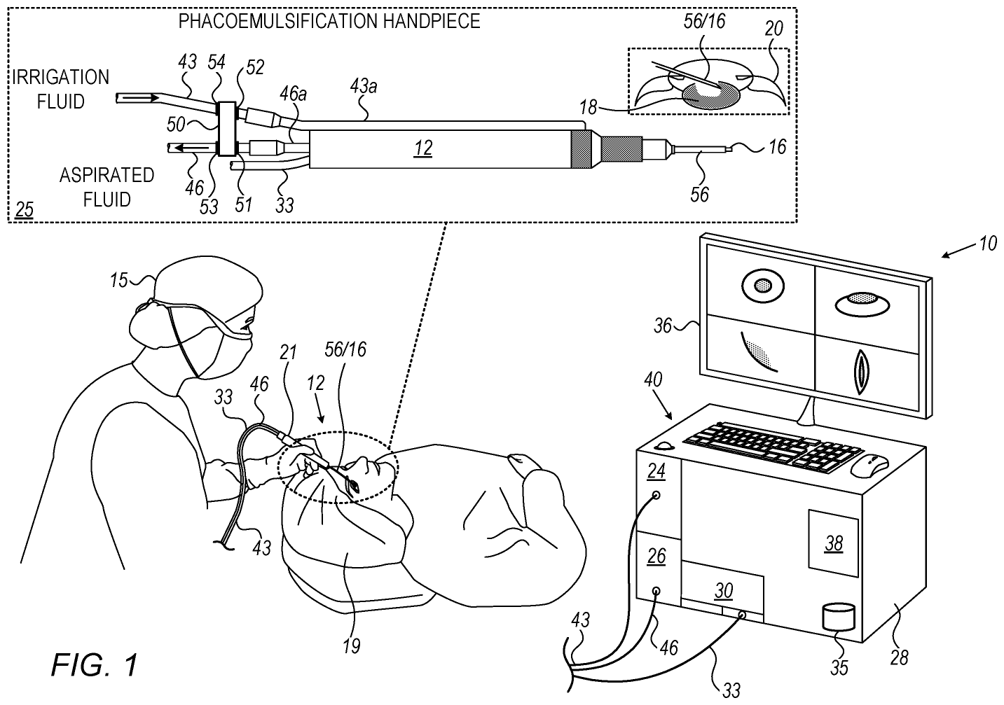

FIG. 1 is a schematic, pictorial view, along with an orthographic side view, of a phacoemulsification apparatus 10 comprising an aspiration and irrigation control module 50 for aspiration and irrigation control, in accordance with an embodiment of the present invention.

As seen in the pictorial view of phacoemulsification apparatus 10, and in inset 25, a phacoemulsification probe 12 (e.g., a handpiece) comprises a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow towards the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Santa Ana, Calif., USA.

In the shown embodiment, during the phacoemulsification procedure, a pumping subsystem 24 comprised in a console 28 pumps irrigation fluid from an irrigation reservoir to the irrigation sleeve 56 to irrigate the eye. The fluid is pumped via an irrigation tubing line 43 running from the console 28 to an irrigation channel 43a of probe 12. Eye fluid and waste matter (e.g., emulsified parts of the cataract) are aspirated via hollow needle 16 to a collection receptacle (not shown) by a pumping subsystem 26, also comprised in console 28, using an aspiration tubing line 46 running from aspiration channel 46a of probe 12 to console 28. In another embodiment, the pumping subsystem 24 may be coupled or replaced with a gravity-fed irrigation source such as a BSS bottle/bag.

Apparatus 10 includes standalone disposable detachable add-on module 50, coupled via fluid connectors 51-54, to control aspiration and irrigation flow rates to reduce risks to eye 20 from irregular performance of aspiration and/or irrigation in probe 12, such as from a vacuum surge. To this end, the disclosed module 50 establishes variable fluid communication between aspiration channel 46a and irrigation channel 43a to control the flow of fluid between the two channels/tubing lines, so as to maintain pressures in the two channels/tubing lines within predefined limits. Moreover, module 50 can discontinue aspiration in parallel in order to provide fast response (e.g., within several milliseconds) to a detect vacuum surge. Module 50 has its own processor and can be used with existing phacoemulsification systems as a disposable element that improves control over intraocular pressure (IOP) during the surgical cataract removal procedure.

Phacoemulsification probe 12 includes other elements (not shown), such as a piezoelectric crystal coupled to a horn to drive vibration of needle 16. The piezoelectric crystal is configured to vibrate needle 16 in a resonant vibration mode. The vibration of needle 16 is used to break a cataract into small pieces during a phacoemulsification procedure. Console 28 comprises a piezoelectric drive module 30, coupled with the piezoelectric crystal, using electrical wiring running in a cable 33. Drive module 30 is controlled by a processor 38 and conveys processor-controlled driving signals via cable 33 to, for example, maintain needle 16 at maximal vibration amplitude. The drive module may be realized in hardware or software, for example, in a proportional-integral-derivative (PID) control architecture.

Processor 38 may receive user-based commands via a user interface 40, which may include setting a vibration mode, duty cycle, and/or frequency of the piezoelectric crystal, and setting or adjusting an irrigation and/or aspiration rate of the pumping subsystems 24/26. In an embodiment, user interface 40 and display 36 may be combined as a single touch screen graphical user interface. In an embodiment, the physician uses a foot pedal (not shown) as a means of control. Additionally, or alternatively, processor 38 may receive the user-based commands from controls located in a handle 21 of probe 12.

Some or all of the functions of processor 38 may be combined in a single physical component or, alternatively, implemented using multiple physical components. These physical components may comprise hard-wired or programmable devices, or a combination of the two. In some embodiments, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 may include further elements which are omitted for clarity of presentation. For example, physician 15 typically performs the procedure using a stereomicroscope or magnifying glasses, neither of which are shown. Physician 15 may use other surgical tools in addition to probe 12, which are also not shown in order to maintain clarity and simplicity of presentation.

### Aspiration Bypass Control in a Phacoemulsification Probe Using Standalone Disposable Detachable Add-on Module

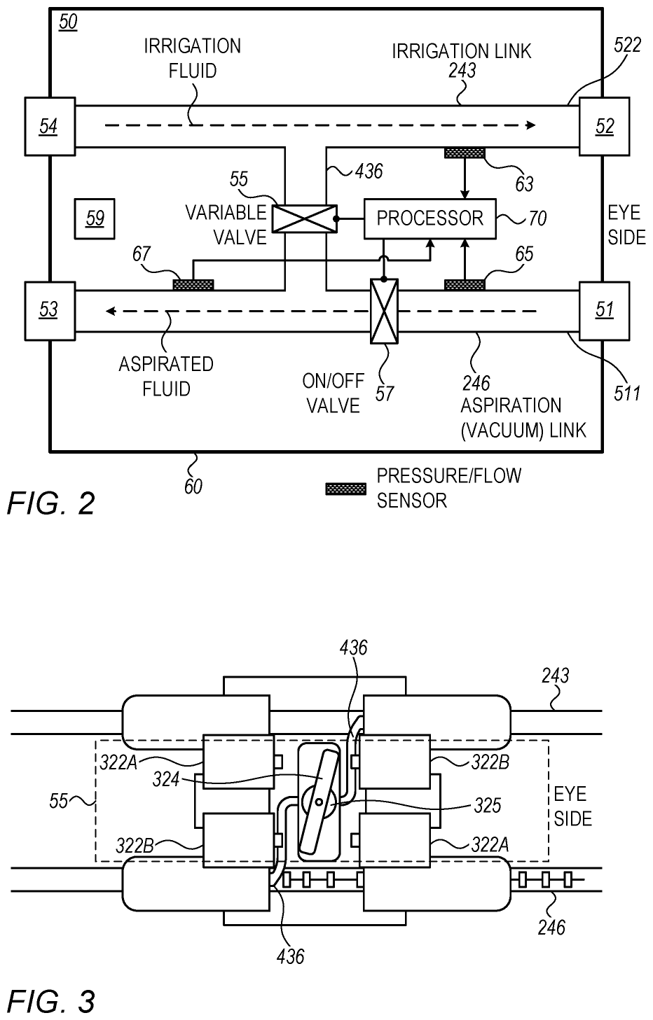

FIG. 2 is a schematic block diagram of the aspiration and irrigation control module 50 of FIG. 1, in accordance with an embodiment of the present invention. In the shown embodiment, standalone module 50 includes a battery 59 inside a package 60 of the module to power a processor, sensors, and electromechanical valves. In general, package 60 includes either an internal power source (e.g., battery 59) or power transfer element (e.g., a socket to plug a power, a wireless power transfer circuit).

As seen, package 60 includes connectors 51-54 fitted on the package that are configured to couple the aspiration and irrigation channels of a probe (46a and 43a via connectors 51 and 52, respectively), and to couple the respective aspiration and irrigation lines of the phacoemulsification system (46 and 43 via connectors 53 and 54, respectively), to the module.

Inside package 60 there is an irrigation link 243 for flowing irrigation fluid from line 43 into irrigation channel 43a, and an aspiration link 246 for removing material from aspiration channel 46a into aspiration line 46. Furthermore, irrigation link 243 is fluidly coupled to aspiration link 246 via a bypass. As seen, a diversion (processor-controlled) variable valve 55 on bypass channel 246 is configured to control a level of fluid communication between irrigation link 243 and aspiration link 246.

An aspiration (processor-controlled) valve 57 is configured to open or close aspiration link 246 at a distal portion of thereof, to, for example, immediately suppress a vacuum surge, until regulated flows are restored by the action of valve 55. While in the shown embodiment valve 57 is an “on/off” valve, in other embodiments it can be a variable valve.

To provide feedback, a sensor 63, such as a pressure sensor or a flow sensor, is coupled to irrigation link 243 to measure the irrigation fluid parameters (e.g., pressure or flow rate) in irrigation link 243 distally to bypass channel 436. A sensor 65, such as a pressure sensor or a vacuum sensor) similarly measures the aspiration sub-pressure in aspiration link 246 distally to bypass channel 436. An additional sensor 67 similarly measures the flow/pressure in aspiration link 246 proximally to bypass channel 436. The pressure/flow and pressure/vacuum measurements are performed close to the aspiration inlet 511 and irrigation outlet 522, respectively, so as to provide an accurate indication of the actual pressures experienced by an eye and provide quick response time to a control loop of module 50.

The term “sensor” includes any sensor type that can provide indications to the processor running module 60. For the aspiration link, such a sensor may be a pressure sensor that is configured to provide sufficiently accurate measurements of low sub-atmospheric pressures that are within a typical range of sub-pressures at which aspiration is applied (e.g., between 1 mm Hg and 650 mm Hg). In an embodiment, sensors 63-67 may comprise the same pressure sensor model, with different settings/calibrations to measure either irrigation pressure or aspiration sub-pressure. For the irrigation link, such a sensor may be the aforementioned pressure sensor, or a fluid flow rate meter.

Based on the fluid pressure/flow measured by sensors 63-67, a processor 70 included in module 50 adaptively adjusts an opening of bypass channel 436 by adjusting diversion valve 55, and in coordination, closing or opening aspiration valve 57. This coordinated operation of the valves maintains pressure/flow readings within predefined limits throughout the surgical procedure, without any dependency on external controls (e.g., of a legacy system to which module 50 is added.

In various embodiments, the different electronic elements of the module shown in FIG. 2 may be implemented using suitable hardware, such as one or more discrete components, one or more Application-Specific Integrated Circuits (ASICs) and/or one or more Field-Programmable Gate Arrays (FPGAs). In some embodiments, the term “processor 70” also includes driving electronics (e.g., high current electronic drivers) required to operate the valves. In other embodiments, the drivers are provided as part of electromechanical valves 55 and 57.

The example module 50 shown in FIG. 2 is chosen purely for the sake of conceptual clarity. For example, other embodiments are possible, such as those that use a smaller number of sensors. As another example, the module may be powered by a power supply external to the module (e.g., via an electrical power socket on the module).

FIG. 3 is a schematic, pictorial cross-sectional view of variable valve 55 of aspiration and irrigation control module 50 of FIG. 2, in accordance with another embodiment of the present invention. Valve 55 is configured to selectively enable fluid communication between irrigation link 243 and aspiration link 246 via bypass channel 436. As seen, diversion valve 55 is a variably rotatable valve comprising a lever 324 coupled to a rotatable valve 325, with pairs 322A and 322B of solenoid pistons arranged, each pair acting alone, to push the lever in one of opposite directions, to generate sufficient torque for valve 325 to adjust the opening of bypass channel 436.

In one embodiment, the two ends of lever 324 are coupled with embedded magnets with the fixed solenoid pairs 322A and 322B on either side of each end of lever 324. With this arrangement, by appropriate selection of the currents that activate either solenoid pair 322A or solenoid pair 322B, a very strong leveraged force (322B clockwise, 322A counterclockwise) can be applied that is capable of overcoming any seizure of valve 325. By switching the solenoid currents the force can be applied to either open or close valve 325. Moreover, by properly selecting solenoid currents, the valve can be rotated into any intermediate location (i.e., partially open to any degree).

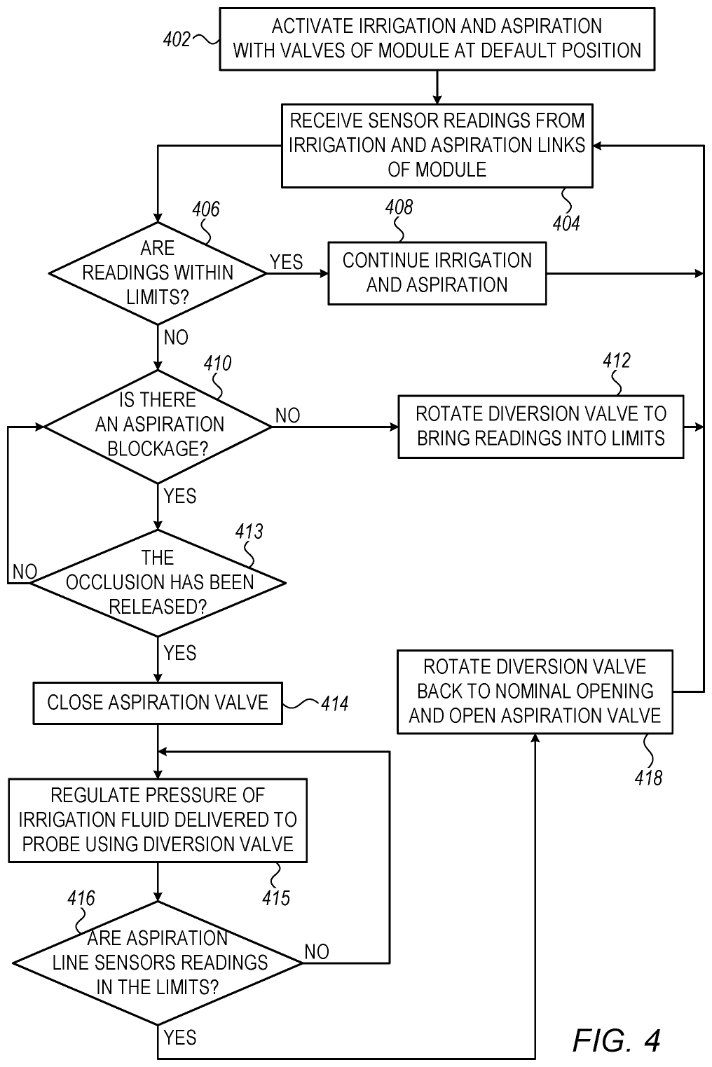

FIG. 4 is a flow chart schematically illustrating a method for overcoming a vacuum surge using aspiration and irrigation control module 50 of FIG. 2, in accordance with an embodiment of the present invention. The algorithm, according to the presented embodiment, carries out a process that begins after physician 15 inserts phacoemulsification needle 16 of probe 12 into a lens capsule 18 of an eye 20.

At a phacoemulsification starting step 402, physician 15 vibrates needle 16 to break-up a cataract and, at the same time, processor 38 activates the aforementioned irrigation and aspiration functions of the probe.

Beforehand, processor 70 verifies (e.g., based on null readings from the sensor upon powering up module 50) the valves 55 and 57 to their default positions (in which valve 57 is open, and valve 55 is closed).

During this process, processor 70 receives pressure readings from irrigation and aspiration links 243 and 246, acquired by sensors 63, 65, and 67, at a sensor reading receiving step 404.

At pressure checking step 406, processor 70 checks if the readings fall within predefined limits. If they do, processor 70 maintains valves 55 and 57 at their default positions, to enable applying the same irrigation and aspiration rate (408) and the process returns to reading step 404.

If the readings do not fall within predefined limits, for example due to unwanted change in irrigation rate, processor 70 checks if the sensor reading(s) indicates the occurrence of a blockage of the aspiration needle, at an aspiration checking step 410. If the readings are not indicative of a blockage or a release of blockage, processor 70 commands diversion valve 55 to rotate in a way that brings the readings back within limits, at a corrective step 412, and the process returns to reading step 404.

If an aspiration blockage occurs, as determined by processor 70 based on, for example, readings from sensor 65 being below predefined values, processor 70 repeatedly checks at a checking step 413 if the occlusion has been released. Once the answer is positive, processor 70 sends signals causing valve 57 to close aspiration link 246 at the distal location, to prevent hazard to the eye from vacuum surge, at an aspiration block response step 414. Valve 57 will be further, or alternatively be, closed when the occlusion is released (this valve has a fast responding time).

Closing valve 57 also increases an irrigation flow via bypass channel 246, which assists in washing away a clog located therein, or a clog at a proximal portion of aspiration link 243.

Then, the processor regulates the pressure (or flow rate, depending, for example on the type of readings from sensors 63, 67) of irrigation fluid in irrigation link 243 by sending signals causing diversion valve 55 to rotate to adjust irrigation fluid flow into aspiration link 246, at an irrigation controlling step 415. Step 415 may also include the option of maintaining bypass channel 436 fully open (“washing mode”) to ensure any vacuum build up in the aspiration line is fully removed from aspiration line 46.

Subsequently (e.g., after a predefined delay time), the processor checks if readings from the aspiration line sensors are within the limits at status checking step 416.

If the answer is no, the processor continues with the irrigation washing or regulation mode, by returning to step 415. If the answer is yes, processor 70 sends a signal causing valve 55 to rotate to a nominal opening or to a closed position, and a signal causes valve 57 to reopen, at a pressure regulation step 418, and the process returns to reading step 404.

The entire checking cycle and application of a corrective action described above, and any of the complete cycles described below, typically may take only several milliseconds, thereby ensuring that no damage is caused to the eye from irrigation and/or aspiration problems.

The example flow chart shown in FIG. 4 is chosen purely for the sake of conceptual clarity. For example, an audiovisual irrigation/aspiration alert may be included, such as when a predefined number of control cycles fail to eliminate a risk of vacuum surge. In such case an audiovisual means may be included in module 50 (e.g., a blinking red light and/or an audio means) and may enter into additional action elements (e.g., activating the audible sound and/or the visual indicator, shutting off one or more pumps, adjusting power delivered to the handpiece, and/or turning off the power to the handpiece, etc.).

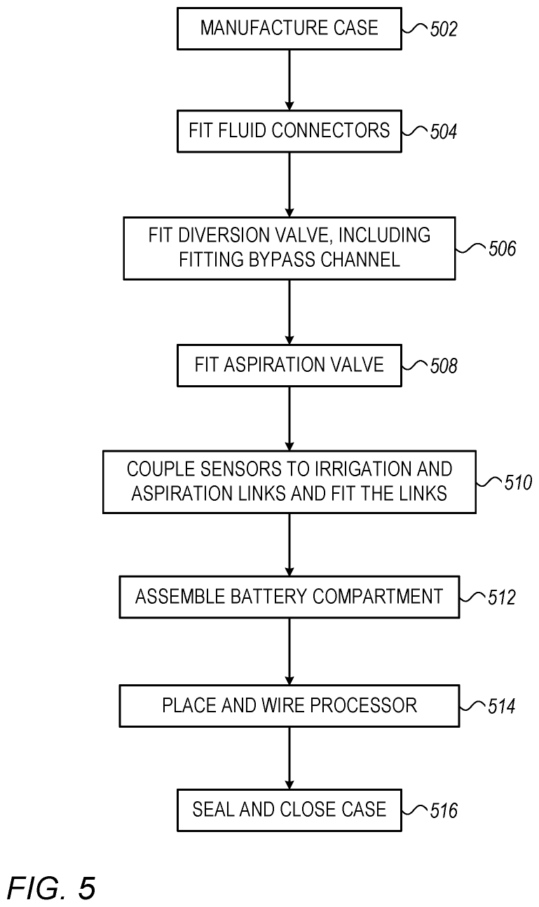

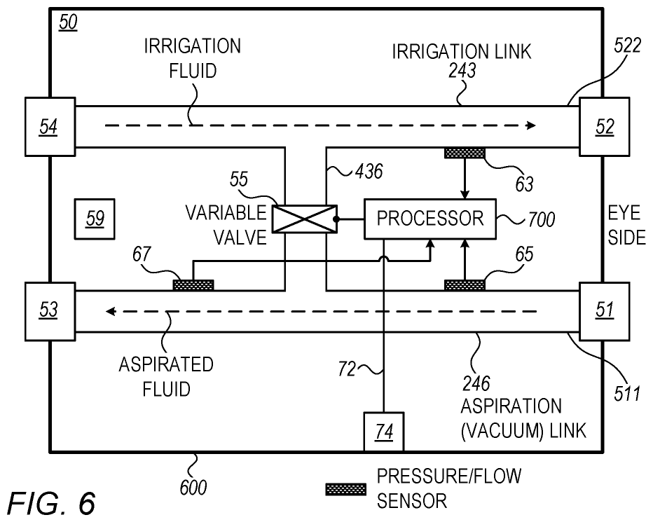

FIG. 5 is a flow chart schematically illustrating a method of manufacturing aspiration and irrigation control module 50 of FIG. 2, in accordance with an embodiment of the present invention.

The process begins with manufacturing package 60, according to a design that enable subsequent assembly steps, at a package manufacturing step 502.

Next, connectors 51-54 are fitted to package 60, at an assembly step 504. Next, diversion valve 55, including bypass channel 436 is placed in position inside package 60, at an assembly step 506.

Next, aspiration valve 57 is placed in position inside package 60, at an assembly step 508.

Next, still unmounted irrigation and aspiration links 243 and 246 are coupled with sensors 63, 65 and 67, at a manufacturing step 508. Then, irrigation and aspiration links 243 and 246 are connected inside the package to aspiration valve 57, to the bypass channel 436 and to connectors 51-54, at channels assembly step 510.

At a battery compartment assembly step 512, a battery compartment including a battery and contacts to the battery is placed inside package 60. At an electronics assembly step 514, processor 70 is placed and wired to the battery contacts, to contacts of sensors 63-67, and to electromechanical valves 55 and 57.

Finally, at a manufacturing step 516, one or more O-rings are placed, e.g., to further isolate electronic elements from fluid piping, and the package is closed and sealed.

The example flow chart shown in FIG. 5 is chosen purely for the sake of conceptual clarity. For example, additional steps, or alternative steps, such as epoxy encapsulations, are omitted for clarity. The order of some of the steps can be changed, as would occur to a person skilled in the art of assembly.

### Another Embodiment of a Disposable Detachable Add-on Module

FIG. 6 is a schematic block diagram of an aspiration and irrigation control module 600, in accordance with another embodiment of the present invention.

In the shown embodiment, standalone module 600 includes a plug 74 and electrical link 72, to connect a processor 700 to console 28, using an external electrical link (not shown), that may be included in cable 33.

As seen, a diversion (processor-controlled) variable valve 55 on bypass channel 436 is configured to control a level of fluid communication between irrigation link 243 and aspiration link 246.

To provide feedback, a sensor 63, such as a pressure sensor or a flow sensor, is coupled with irrigation link 243 to measure the irrigation fluid pressure (or flow rate) in irrigation link 243 distally to bypass channel 436. A sensor 65 (such as a pressure sensor or a vacuum sensor) similarly measures the aspiration sub-pressure in aspiration link 246 distally to bypass channel 436. An additional sensor 67 similarly measures the flow/pressure in aspiration link 246 proximally to bypass channel 436. The pressure/flow and pressure/vacuum measurements are performed close to the aspiration inlet 511 and irrigation outlet 522, respectively, so as to provide an accurate indication of the actual pressures experienced by an eye and provide quick response time to a control loop of module 600.

Based on the fluid pressure/flow measured by sensors 63-67, processor 700 included in module 50 adaptively adjusts an opening of bypass channel 436 by adjusting diversion valve 55, and in coordination, may command using the electrical line to the console, turning off the ultrasound to the emulsifying needle, to prevent overheating of the eye, and may also increase the irrigation rate to compensate for the diverted irrigation fluid. In an alternative embodiment, processor 700 maintains diversion valve 55 opened, irrigation rate is increased (by the irrigation pump command) to compensate for fluid flowing into bypass channel 436 and US power is applied (even if momentarily) to break up the material causing the occlusion. In an embodiment, processor 700 commands a boost of US power delivered to the probe and only afterwards the ultrasound is shut off, or US power is lowered.

When processor 700 detects the occlusion has cleared, the processor closes the diversion valve and turns the emulsifying needle back on. By not having any valve in the aspiration line, there is no possibility of such a valve functioning incorrectly.

This coordinated operation of the valves maintains pressure/flow readings within predefined limits throughout the surgical procedure.

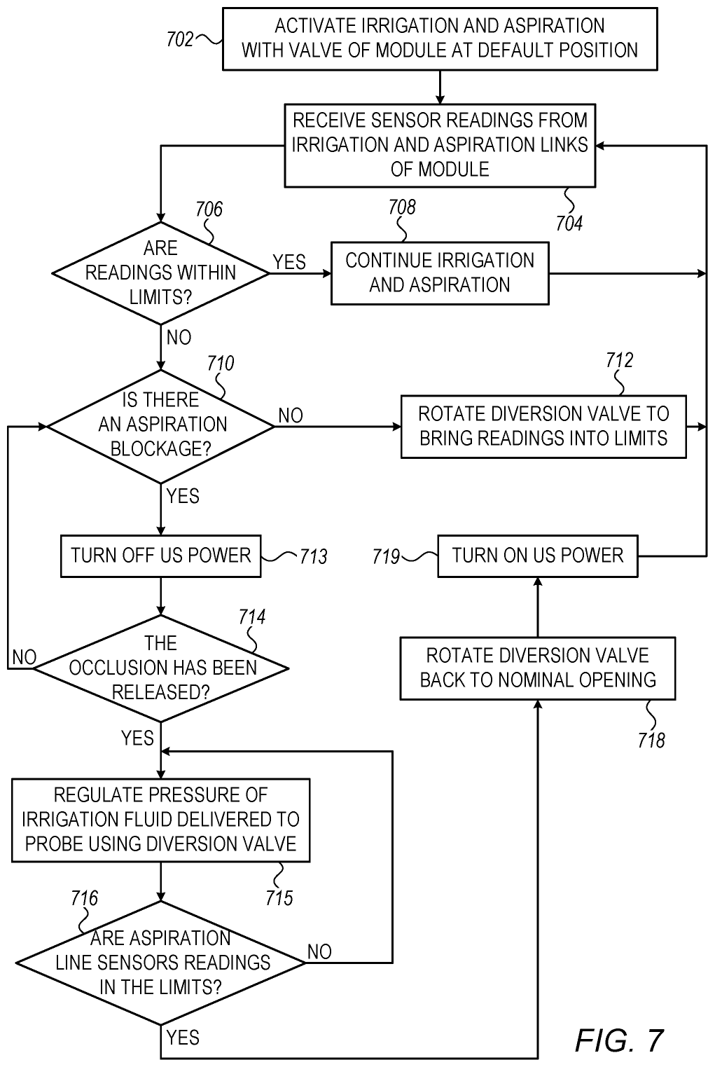

FIG. 7 is a flow chart schematically illustrating a method for overcoming a vacuum surge using aspiration and irrigation control module 600 of FIG. 6, in accordance with another embodiment of the present invention.

The algorithm, according to the presented embodiment, carries out a process that begins after physician 15 inserts phacoemulsification needle 16 of probe 12 into a lens capsule 18 of an eye 20.

At a phacoemulsification starting step 702, physician 15 vibrates needle 16 to break-up a cataract and, at the same time, processor 38 activates the aforementioned irrigation and aspiration functions of the probe. Beforehand, processor 700 verifies (e.g., based on null readings from the sensor upon powering up module 50) the valve 55 is closed.

During this process, processor 700 receives pressure readings from sensors 63, 65, and 67 of irrigation and aspiration links 243 and 246 at a sensor reading receiving step 704.

At pressure checking step 706, processor 700 checks if the readings fall within predefined limits. If they do, processor 70 maintains the valve in a closed position, to enable applying the same irrigation and aspiration rate (708) and the process returns to reading step 704.

If the readings do not fall within predefined limits (e.g., due to an unwanted change in irrigation rate), processor 700 checks if the sensor reading(s) indicates the occurrence of a blockage of the aspiration needle, at an aspiration checking step 710. If the readings are not indicative of a blockage or a release of a blockage, processor 700 may command diversion valve 55 to rotate in a way that brings the readings back within limits, at a corrective step 712, and the process returns to reading step 704. Additionally, or alternatively, step 712 may comprise one or more of the steps of adjust the irrigation pump rate, changing the irrigation bottle height, then using the valve if the irrigation rate is too high.

If an aspiration blockage occurs, as determined by processor 700 based on, for example, readings from sensor 65 being below predefined values, processor 700 may command adjusting the US power (e.g., increasing it, lowering it, and/or shutting off of US power) and/or changing a vibration mode (e.g., longitudinal, transverse, and/or torsional vibrational modes that define each, or in combination, a given vibration trajectory of needle 16), frequency, and/or duty cycle of needle 16, at an optional US power shutting down step 713. Next, processor 700 repeatedly checks at a checking step 714 if the occlusion has been released.

Once the answer is positive, the processor regulates the pressure (or flow rate, depending, for example on the type of readings from sensors 63, 67) of irrigation fluid in irrigation link 243 by sending signals causing diversion valve 55 to rotate to adjust irrigation fluid flow into aspiration link 246, at an irrigation controlling step 715. Step 715 may also include the option of maintaining bypass channel 436 in a fully opened position (“washing mode”) to ensure any vacuum build up in aspiration channel 46a is eliminated.

Subsequently (e.g., after a predefined delay time), the processor checks if aspiration line sensor readings are in the limits at a status checking step 716.

If the answer is no, the processor continues with the irrigation washing or regulation mode, by returning to step 715. If the answer is yes, processor 700 sends a signal causing valve 55 to rotate to a nominal opening or a closed position, at a pressure regulation step 718. Next, processor 700 commands the turning on of US power to needle 16, at an US power upping step 719, and the process returns to reading step 704.

The example flow chart shown in FIG. 7 is chosen purely for the sake of conceptual clarity. For example, an audiovisual irrigation/aspiration alert may be included, such as when a predefined number of control cycles fail to eliminate a risk of vacuum surge. In such case an audiovisual means that may be included in module 600 (e.g., a blinking red light and/or an audio means) may enter into action.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A module for controlling irrigation and aspiration of a phacoemulsification probe inserted into an eye, the module comprising:
an irrigation link, configured to be coupled with an irrigation line and an irrigation channel of the phacoemulsification probe;
an aspiration link, configured to be coupled with an aspiration line and an aspiration channel of the phacoemulsification probe;
a bypass channel coupled with the irrigation link and the aspiration link to enable diversion of irrigation fluid from the irrigation link to the aspiration link;
an aspiration valve coupled with the aspiration link and configured to regulate fluid flow via the aspiration link;
a diversion valve coupled with the bypass channel and configured to regulate fluid flow from the irrigation link to the aspiration link;
a first sensor coupled with the irrigation link and a second sensor coupled with the aspiration link, wherein the first sensor and second sensor are configured to measure fluid parameters in the irrigation link and in the aspiration link; and
a processor in communication with the first sensor and the second sensor, wherein the processor is configured to identify a change in at least one of the fluid parameters by reading at least one of the first sensor and the second sensor, and, in response to the identified change in the at least one of the fluid parameters, (i) close the aspiration valve and (ii) maintain a pressure of the irrigation fluid delivered to the irrigation channel within a predefined range, by regulating the fluid flow via the bypass channel using the diversion valve.

2. The module according to claim 1, wherein the at least one of the fluid parameters are selected from the group consisting of vacuum, pressure, and flow, and the change of the at least one of the fluid parameters indicates an aspiration blockage or a release of the aspiration blockage.

3. The module according to claim 1, further comprising connectors for detachably connecting the irrigation line and the aspiration line to the phacoemulsification probe.

4. The module according to claim 1, further comprising a package containing the irrigation link, the aspiration link, the bypass channel, the aspiration valve, the diversion valve, the sensors and the processor.

5. The module according to claim 1, wherein the processor is configured to identify the change in the at least one of the fluid parameters being an increase in vacuum or pressure in the aspiration line or aspiration channel.

6. The module according to claim 1, wherein the processor is configured to adjust the diversion valve so as to maintain a pressure level in the irrigation channel within a predefined limit.

7. The module according to claim 1, wherein the first sensor comprises a pressure sensor coupled with the irrigation link distally to the bypass channel, the second sensor comprises a vacuum sensor coupled with the aspiration link distally to the bypass channel, and wherein the module further comprises a third sensor, wherein the third sensor is a flow sensor coupled with the aspiration link proximally to the bypass channel.

8. The module according to claim 1, wherein the diversion valve is a variably rotatable valve comprising (i) a lever coupled with the diversion valve, and (ii) one or more solenoid pistons that, when actuated by the processor, moves the lever so as to generate rotational torque causing the diversion valve to adjust an opening in the bypass channel.

9. A module for controlling irrigation and aspiration of a phacoemulsification probe inserted into an eye, the module comprising:
an irrigation link, configured to be coupled with an irrigation line and an irrigation channel of the phacoemulsification probe;
an aspiration link, configured to be coupled with an aspiration line and an aspiration channel of the phacoemulsification probe;
a bypass channel coupled with the irrigation link and the aspiration link to enable diversion of irrigation fluid from the irrigation link to the aspiration link;
a diversion valve coupled with the bypass channel and configured to regulate fluid flow from the irrigation link to the aspiration link;
a first sensor coupled with the irrigation link and a second sensor coupled with the aspiration link, wherein the first sensor and second sensor are configured to each measure a fluid parameter in the irrigation link and in the aspiration link; and
a processor in communication with the first sensor and the second sensor, and in communication with an ultrasonic power source of the phacoemulsification probe, wherein the processor is configured to identify a change in at least one of the fluid parameters by reading at least one of the first sensor and the second sensor, and, in response to the identified change in the at least one of the fluid parameters, (i) activate the diversion valve to regulate the fluid flow via the bypass channel and (ii) adjust the ultrasonic power source of the phacoemulsification probe.

10. The module according to claim 9, wherein the at least one of the fluid parameters are selected from the group consisting of vacuum, pressure, and flow, and the change of the at least one of the fluid parameters indicates an aspiration blockage or a release of the aspiration blockage.

11. The module according to claim 9, wherein the processor is configured to adjust the ultrasonic power by shutting off the power.

12. The module according to claim 9, wherein the processor is configured to adjust the ultrasonic power by changing one or more selected from the group consisting of frequency, duty cycle, and vibration mode of the probe.

13. The module according to claim 9, further comprising connectors for detachably connecting the irrigation line and the aspiration line to the phacoemulsification probe.

14. The module according to claim 9, further comprising a package containing the irrigation link, the aspiration link, the bypass channel, an electrical link for communication with the ultrasonic power source, the diversion valve, the sensors and the processor.

15. The module according to claim 9, wherein the processor is configured to identify the change in aspiration, and to control the diversion valve.

16. The module according to claim 9, wherein the processor is configured to adjust the diversion valve so as to maintain a pressure level in the irrigation channel within a predefined limit.

17. The module according to claim 9, wherein the first sensor comprises a pressure sensor coupled with the irrigation link distally to the bypass channel, the second sensor comprises a vacuum sensor coupled with the aspiration link distally to the bypass channel, and wherein the module further comprises a third sensor, wherein the third sensor is a flow sensor coupled with the aspiration link proximally to the bypass channel.

18. The module according to claim 9, wherein the diversion valve is a variably rotatable valve comprising (i) a lever coupled with the diversion valve, and (ii) one or more solenoid pistons that, when actuated by the processor, moves the lever so as to generate rotational torque causing the diversion valve to adjust an opening in the bypass channel.

19. A method of controlling irrigation and aspiration of a phacoemulsification probe inserted into an eye, the method comprising:
providing a module configured to control the irrigation and aspiration of the phacoemulsification probe, wherein the module comprises:
an irrigation link, configured to be coupled with an irrigation line and an irrigation channel of the phacoemulsification probe;
an aspiration link, configured to be coupled with an aspiration line and an aspiration channel of the phacoemulsification probe;
a bypass channel coupled with the irrigation link and the aspiration link to enable diversion of irrigation fluid from the irrigation link to the aspiration link;
an aspiration valve coupled with the aspiration link and configured to regulate fluid flow via the aspiration link;
a diversion valve coupled with the bypass channel and configured to regulate fluid flow from the irrigation link to the aspiration link;
a first sensor coupled with the irrigation link and a second sensor coupled with the aspiration link, wherein the first sensor and second sensor are configured to measure fluid parameters in the irrigation link and in the aspiration link; and
a processor in communication with the first sensor and the second sensor, wherein the processor is configured to identify a change in at least one of the fluid parameters; and
reading at least one of the first sensor and the second sensor to identify a change in the at least one of the fluid parameters; and
in response to the identified change in the at least one of the fluid parameters, (i) closing the aspiration valve, and (ii) maintaining a pressure of the irrigation fluid delivered to the irrigation channel within a predefined range, by regulating the fluid flow via the bypass channel using the diversion valve.

20. The method according to claim 19, wherein the at least one of the fluid parameters are selected from the group consisting of vacuum, pressure, and flow, and the identified change of the at least one of the fluid parameters indicates an aspiration blockage or a release of the aspiration blockage.

21. The method according to claim 19, wherein the identified change in the at least one of the fluid parameters is an increase in vacuum or pressure in the aspiration line or aspiration channel.

22. The method according to claim 19, wherein the diversion valve is adjusted so as to maintain a pressure level in the irrigation channel within a predefined limit.

23. The method according to claim 19, wherein the first sensor is a pressure sensor and is coupled with the irrigation link distally to the bypass channel, the second sensor is a vacuum sensor and is coupled with the aspiration link distally to the bypass channel, and wherein the module further comprises a third sensor, wherein the third sensor is a flow sensor, and is coupled with the aspiration link proximally to the bypass channel.

24. A method of controlling irrigation and aspiration of a phacoemulsification probe inserted into an eye, the method comprising:
providing a module configured to control the irrigation and aspiration of the phacoemulsification probe, wherein the module comprises:
an irrigation link, configured to be coupled with an irrigation line and an irrigation channel of the phacoemulsification probe;
an aspiration link, configured to be coupled with an aspiration line and an aspiration channel of the phacoemulsification probe;
a bypass channel coupled with the irrigation link and the aspiration link to enable diversion of irrigation fluid from the irrigation link to the aspiration link;
a diversion valve coupled with the bypass channel and configured to regulate fluid flow from the irrigation link to the aspiration link;
a first sensor coupled with the irrigation link and a second sensor coupled with the aspiration link, wherein the first sensor and second sensor are configured to each measure a fluid parameter in the irrigation link and in the aspiration link; and
a processor in communication with the first sensor and the second sensor, and in communication with an ultrasonic power source of the phacoemulsification probe, wherein the processor is configured to identify a change in at least one of the fluid parameters; and
reading at least one of the first sensor and the second sensor to identify a change in the at least one of the fluid parameters; and
in response to the identified change in the at least one of the fluid parameters, (i) activating the diversion valve to regulate the fluid flow via the bypass channel, and (ii) adjusting the ultrasonic power source of the phacoemulsification probe.

25. The method according to claim 24, wherein the at least one of the fluid parameters are selected from the group consisting of vacuum, pressure, and flow, and the identified change of the at least one of the fluid parameters indicates an aspiration blockage or a release of the aspiration blockage.

26. The method according to claim 24, wherein adjusting the ultrasonic power comprises shutting off the power.

27. The method according to claim 24, wherein adjusting the ultrasonic power comprises changing one or more selected from the group consisting of frequency, duty cycle, and vibration mode of the probe.

28. The method according to claim 24, wherein identifying the change comprises identifying a change in aspiration, and controlling the diversion valve.

29. The method according to claim 24, wherein adjusting the diversion valve comprises maintaining a pressure level in the irrigation channel within a predefined limit.

30. The method according to claim 24, wherein the first sensor is a pressure sensor and is coupled with the irrigation link distally to the bypass channel, the second sensor is a vacuum sensor and is coupled with the aspiration link distally to the bypass channel, and wherein the module further comprises a third sensor, wherein the third sensor is a flow sensor, and is coupled with the aspiration link proximally to the bypass channel.
